(19) United States
(12) Patent Application Publication
(10) Pub. No.: US 2018/0294054 A1
(43) Pub. Date: Oct. 11, 2018

(54) METHOD AND DEVICE FOR PRODUCING A SERIES OF IMAGES OF AN OBJECT

(71) Applicant: FOTOFINDER SYSTEMS GMBH, Bad Birnbach (DE)

(72) Inventor: Andreas MAYER, Passau (DE)

(21) Appl. No.: 15/575,706

(22) PCT Filed: Apr. 6, 2016
(86) PCT No.: PCT/EP2016/057485

§ 371 Date: Nov. 20, 2017

(30) Foreign Application Priority Data
May 27, 2015 (DE) 10 2015 108 355.8

(51) Int. Cl. G16H 30/20; G06F 17/30; G16H 40/63

(52) U.S. Cl. CPC G16H 30/20; G16H 40/63; G06F 17/3028

(57) ABSTRACT

The invention relates to a method and a device for generating a series of images of an object, each having predetermined image parameters, by means of a partly automated imaging system (02) comprising a databank, an imaging unit (06), a computing unit (10), a storage unit (16) and an output unit (12), it being ensured that a image parameter to be set manually correlates to an actual image parameter.

The invention relates to a method for generating a series of images of an object as well as a corresponding imaging system for generating a series of images of an object.

Such systems and methods are used in the field of medical treatments or surgical interventions, for example. In this context, the methods and the devices serve for controlling and/or documenting the treatment's progress and/or the results of surgical interventions. A series of images can be generated using generic devices and methods prior to a corresponding surgical intervention, for example, said images showing the body in treatment holding different postures and/or in different spatial orientations with respect to an imaging unit, with one or more corresponding series of images being generated subsequent to the surgical intervention in order to document the healing process and/or the success of the surgical intervention.

Corresponding devices and methods can also be used for documenting the treatment of a body for which a surgical intervention is not required. Thus, aesthetically indicated treatments of a body can, for example, be administered without surgical intervention, a documentation of the initial state of the body before commencing the treatment as well as the further development of the body after termination of the treatment being of particular interest here. An example is the removal of tattoos by means of laser radiation, for example.

In the scope of the invention, the objects to be imaged are to be, overall, human or animal bodies. Moreover, the series of images of the human or animal bodies, as disclosed in the following, are generated for documenting medical or aesthetic interventions and/or treatments of the human or animal body.

The series of images produced thus or the comparison of images from different picture series and the informative value resulting from the comparison regarding the treatment's success depends highly on whether the corresponding images of the different picture series were recorded using correlating image parameters. In known systems and methods, for example, output units are provided by means of which image parameters to be set manually of objects are output for a user so as to generate identical or reproducible properties of the images of the picture series.

The known output of image parameters to be set manually of the imaging system, however, does not prevent images or entire picture series from being recorded or generated using wrong image parameters.

Based on this state of the art, it is therefore the object of the present invention to propose a method as well as a system by means of which recording or generating images of picture series using incorrect image parameters to be set manually is avoided.

This object is attained by means of the method of the invention according to claim 1 as well as the system of the invention according to claim 8.

Advantageous embodiments of the method as well as of the system are the subject matter of the dependent claims.

The method serves for generating a series of images of an object by means of a partly automated imaging system using predetermined image parameters in each instance, said imaging system comprising a databank, an imaging unit, a computing unit, a storage unit and an output unit. For this purpose, the method comprises the following method steps.

In a first method step, a picture series presetting comprising a plurality of predefined groups of image parameters of an object is selected from a databank. A corresponding picture series presetting can also be called a template. A certain part of the body to be imaged, a certain spatial orientation of the body to be imaged regarding the imaging unit, a certain posture of the body to be imaged as well as a plurality of parameters of an imaging unit, such as exposure time/ISO value, focal length, flash properties and much more, can be deposited or stored as an image parameter for the corresponding group of a picture series presetting, as an by no means limiting example.

As previously described, the method according to the invention is used in order to make a comparison based on a first imaging series to other corresponding picture series recorded at a later stage. Accordingly, the method can be used for generating a basic picture series and for later generating follow-up picture series. In the case of a basic picture series, the first method step can also be designed such that selection of the picture series presetting is stored. A selection of the picture series can then be intended merely indirectly on the basis of the stored picture series presetting when executing the method for generating a follow-up picture series. In other words, this means that the first method step can also comprise an input which determines whether a basic picture series or a follow-up picture series is to be generated, said picture series presetting to be accordingly selected indirectly in a basic picture series or indirectly in a follow-up picture series.

In a further method step, at least one image parameter to be manually set of the corresponding group of the picture series presetting is output on the output unit. A screen can serve as an output unit, for example, said screen being configured for generating and outputting visualizations of the image parameters to be set manually in collaboration with a computing unit if necessary. Alternatively, an acoustic output unit can be provided as an output unit, such as a loudspeaker for example. By means of the thus generated output, the user is informed of the image parameters which cannot be set within the system in the scope of the partly automated imaging system, but instead require being manually set.

In a following method step, which is, for example, initiated by the user verifying the settings of the image parameters to be set manually by means of a corresponding input into an input unit of the system, an image is generated by means of the imaging unit. The imaging unit can be a digital imaging unit, for example, comprising a digital image sensor and focusing optics. In this context, it can be intended that the imaging system is operated using a plurality of commercially produced imaging units, such as mass-produced digital single-lens reflex cameras.

Subsequent to generating the image, the image is transmitted to the computing unit by means of suitable devices of the system. The transmission can be a wireless or wired transmission of digital picture data or digital imaging data.

In the following method step, the image is evaluated with respect to at least one manually set image parameter by means of the computing unit. For this purpose, the computing unit can analyze or evaluate the image or picture data in different manners. On the one hand, it can be possible to evaluate the actual picture data in the form of their individual pixels and their arrangement to each other. On the other hand, data can also be considered in the evaluation which are generated by the imaging unit and are transmitted with the imaging data, which directly or indirectly concern the image parameters of the imaging unit occurring in the imaging. In this manner, common imaging units also generate data on the settings of the imaging unit for each recorded image, for example, said settings not being reflected in the corresponding imaging itself, such as the focal length of a focusing optics of the imaging unit as part of the picture data, for example.

Subsequent to the evaluation, a notification is put out by means of the output unit in another method step in the event that the preceding evaluation on the part of the computing unit yields or rather has yielded that, in the corresponding preceding method step, the at least one manually set image parameter does not correlate to the output of the image parameter to be set. The evaluation of the computing unit can, for example, draw the conclusion that a certain focal length of the imaging unit is predetermined for the group of picture series presetting at hand and that this focal length was output as an image parameter to be set manually, said analyzed or rather evaluated image or rather the underlying picture data, however, allowing to directly or indirectly draw conclusions on a deviant focal length of the imaging unit. The deviation can be brought to the user's attention by putting out a notification via the output unit as intended.

The afore-described method steps are thus applied for each group of the picture series presetting, starting with the method step in which at least one image parameter to be manually set is output.

In the event that the at least one manually set image parameter does not correlate to the output image parameter to be set, the method enables, as has become clear in the described individual method steps, that the groups of images from the corresponding picture series presetting are generated with identical image parameters, in particular with respect to the image parameters to be set manually, during each processing, such as before and after treating a body to be imaged by evaluating the image via the computer unit and the following notification, whereby the reproducibility of the images and thus the informative value of the images' comparison from different picture series is significantly increased.

It can be intended in a further development of the method that the image is saved by means of a storage unit in an additional method step. For this purpose, it can either be intended that the saving process only takes place when the evaluation performed by the computing unit has not yielded a deviation regarding the presetting and the actual image parameters to be set manually. Alternatively, however, it can also be intended that the saving process takes place in such manner that a detected deviation of the imaging parameters to be set manually from the presetting is also saved. Thus, it can be ensured that either only images having correct imaging parameters to be set manually are saved and are correspondingly available for documentation or that the detected deviation is also documented in the documentation and the comparison based thereon of the images and, in so doing, consequently preventing an erroneous informative value of the result via the comparison of the images.

Moreover, it can be intended that the method comprises an additional method step which succeeds the step of evaluating the image by means of the computing unit and intends to repeat the preceding method steps, starting with the method step of outputting at least one image parameter to be manually set for the corresponding group of the picture series presetting, in the event that the evaluation yields that the at least one image parameter to be manually set does not correlate to the output of the corresponding image parameter in the preceding method step. This has the advantage of thus ensuring that an image, in which the predetermined manually settable image parameters are actually present, is generated for each group of the picture series presetting and thus an image being able to be generated and saved, which leads to a conclusive result when comparing a corresponding image of a previous and/or later image from the same group of the picture series presetting.

In another embodiment of the method, it can be intended that storing the image in a storage unit comprises automatically generating a filename and/or metadata of the image. Similarly, the file location can be automated by referencing the data to be stored of the image accordingly. It can be intended, for example, that the filename and/or the metadata is/are automatically generated on the basis of the picture series presetting, the corresponding groups of the picture series presetting, an object description, a date or on the basis of various other information accessible by the system and/or image parameters.

Moreover, it is advantageous if the method comprises another method step which is integrated between outputting at least one image parameter to be set manually and generating the corresponding image and intends using a setting device, an infinitely settable image parameter of the imaging unit being able to be manually set stepwise by means of the setting device. The focal length of focusing optics of the imaging units can be, for example, manually and infinitely set in a large majority of commercially produced imaging devices or focusing optics. The focal length can be gradually set by means of the proposed setting device, which is arranged temporarily or permanently on the imaging unit and/or the focusing optics of the imaging unit. Such a use of a setting device has the advantage of the predetermined image parameters to be manually set of the imaging unit being able to set correctly, and in particular without slight variations, by the user.

In another embodiment of the method, it is advantageously intended that at least one remotely set image parameter can be transmitted from the databank to a unit of the imaging system, in particular to the imaging unit and a corresponding setting of the image parameter on the side of the corresponding unit of the imaging system, in a method step which is switched before the step of generating an image. The method for partly automated systems is adjusted particularly advantageously via the proposed transferal and setting. In other words, this means that the imaging system is remotely set via corresponding interfaces or automatically set to the corresponding image parameters whenever possible by means of the method and that the manual setting of image parameters along with the initial output and the evaluation of the actual manual setting is carried out only for those image parameters, which inevitably require a manual setting by the user, by means of the feedback described above or rather the feedback loop. Thus, an illumination time for the image can be, for example, sent to the imaging unit via a corresponding interface between the databank and the imaging unit and can, accordingly, be set automatically by said imaging unit.

In another advantageous embodiment, the initial method step of selecting a picture series presetting also comprises selecting object data from another databank and/or inputting object data into an input unit of the imaging system, the image parameters of the picture series presetting being adjusted in dependence of the object data. Object data, such as height, sex, skin type, weight, can be stored or rather input into the other databank or via the input unit, respectively. For this purpose, it can be intended that the image parameters are adjusted for a picture series presetting or rather its groups based on individual or several such object data on the basis of suitable algorithms on the part of the computing unit. Thus, it becomes possible that all image parameters, in particular the image parameters to be set manually as well, are ideally adjusted to the object to be imaged, whereby the informative value can also be further improved via the comparison of correlating groups of images.

In this instance, it can also be intended that the image parameters are adjusted when executing the method for generating a basic picture series and that the already adjusted and possibly saved image parameters are adopted when generating a follow-up picture series at a later time. Alternatively, however, an adjustment can be intended whenever the method is executed. This allows, for example, considering changes in the pigmentation of the skin of the object to the imaged.

In another embodiment, moreover, it can be intended that the initial method step of selecting a picture series presetting via a corresponding combination of output steps and input steps by means of the corresponding input unit and output unit enables activating or deactivating, respectively, individual groups of image parameters of a picture series presetting, the following method steps only being executed for the activated groups of image parameters of the picture series presetting. In other words, this means that it becomes possible for the user to limit the number of images and consequently the groups of image parameters of a picture series presetting to the logical or necessary groups via a corresponding input and a selection resulting therefrom. Thus, for example, a group of a picture series presetting, for which a certain spatial orientation of the object regarding the imaging unit is intended which the user deems as illogical or uninformative either in its entirety or based on circumstances specific to the object, can be deactivated in order to not unnecessarily enlarge the entire method or to limit it to informative images.

Such a change in the picture series presetting can be particularly advantageous when executing the method for generating a basic picture series and are saved or deposited in such a manner that the changes are preserved when subsequently executing the method for generating follow-up picture series.

Moreover, it is advantageous if a new or individual picture series presetting can be generated in the initial method step. In this context, it can be intended, on the one hand, that the picture series presetting is entirely regenerated. On the other hand, however, it can be intended that the picture series presetting is generated by altering an already existing picture series presetting. For this purpose, new picture series presettings can also be changed or altered via corresponding inputs and outputs at the input unit and the output unit, for example by using an editor of a graphic user surface.

Advantageously, the generation is intended only globally or when executing the method for generating a basic picture series when correspondingly adopting the generated picture series presetting for the subsequent execution of the method.

The imaging system according to the invention for generating a series of images of an object each having predetermined image parameters comprises, as described above, a databank, an imaging unit, a computing unit, a storage unit and an output unit.

In this context, the databank is configured for storing picture series presettings comprising a plurality of predefined groups of image parameters of an object. The input unit is intended for selecting a picture series presetting. Furthermore, the imaging unit is configured for generating images of the object and for transferring the images to the computing unit and/or the storage unit, and the storage unit is configured for storing the generated images. Moreover, the output unit is configured for outputting at least one image parameter to be set manually of the corresponding group of a selected picture series presetting, the computing unit is moreover configured for evaluating an image with respect to at least one image parameter to be set manually, and the output unit is configured for outputting a notification in case the evaluation by means of the computing unit yields that the at least one image parameter to be set manually does not correlate to the output of the corresponding image parameter on the output unit.

The proposed imaging system thus enables determining that the image parameters to be set manually were set correctly for each generated image before the corresponding image was generated. Accordingly, using the proposed imaging system when comparing correlating images from different picture series, the informative value can be increased by the images allocated to a corresponding group from different picture series having identical image parameters to be set manually or rather having identical image parameters to be set manually being generated.

Furthermore, it is particularly advantageous if the computing unit is configured for generating filenames and/or metadata in dependence of a picture series presetting, the group of the picture series presetting in dependence of object data available in a different databank and/or in dependence of information input into an input unit and for storing the aforementioned with the images in the storage unit.

Furthermore, it is advantageously intended that the imaging system comprises a setting device which is arranged at the imaging unit and enables a stepwise setting of an image parameter which can be infinitely set by means of the imaging unit. As previously described above, such a setting device enables that the image parameters infinitely and manually set at the imaging unit are set precisely and in particular without any slight deviances.

For this purpose, it is particularly advantageous if the setting device is arranged at the imaging unit in such a manner that a stepwise setting of the focal length at a focusing optics of the imaging unit having an infinitely settable focal length becomes possible. A setting device can, for example, comprise two ring elements, which are arranged concentrically to each other and are mounted so as to be rotatable to each other and comprise corresponding means for being arranged or fixed, respectively, to the imaging unit and/or the focusing optics of the imaging unit.

Furthermore, interacting locking elements can be intended on the two ring elements, said locking elements enabling detachably locking the two ring elements in a defined relative arrangement regarding a rotational axis defined by the shared middle axis. Furthermore, a defined arrangement regarding the imaging unit and/or the focusing optics can be attained by means of the fixing means in order to ensure that the positions settable in a stepwise manner of the ring elements to each other correlate to an image parameter to be set manually, in particular having a determined focal length.

Moreover, it is particularly advantageous if the imaging system comprises at least one transmitting means by means of which at least one remotely settable image parameter is transmitted to a unit of the imaging system, in particular to the imaging unit. Via the proposed transmitting means, which can be formed, for example, as a combination of interfaces and/or cable connections, it can be ensured that all remotely settable image parameters are automatically set in the partly automated imaging system and that thus errors are prevented which would reduce the comparability of the resulting images from different picture series of the same object. Preventing errors regarding the image parameters is moreover improved by evaluating the images in regard of the image parameters to be set manually of the imaging systems.

In this context, it is further particularly advantageous if the imaging system comprises at least one setting means for remotely setting an image parameter, said setting means being connected to a transmitting means in order to receive the corresponding remotely settable image parameter. As previously described, the interaction between the transmitting means and the corresponding setting means enables that individual units of the imaging system are set at least partially remotely to the corresponding intended image parameter.

Additionally, it can be advantageously intended for the computing unit to be configured for adjusting the image parameters of a picture series presetting by means of object data of the object to be imaged.

In the following, individual embodiments of the method as well as of the system will be described in an exemplary manner by way of the only schematically illustrated drawing. In the drawing,

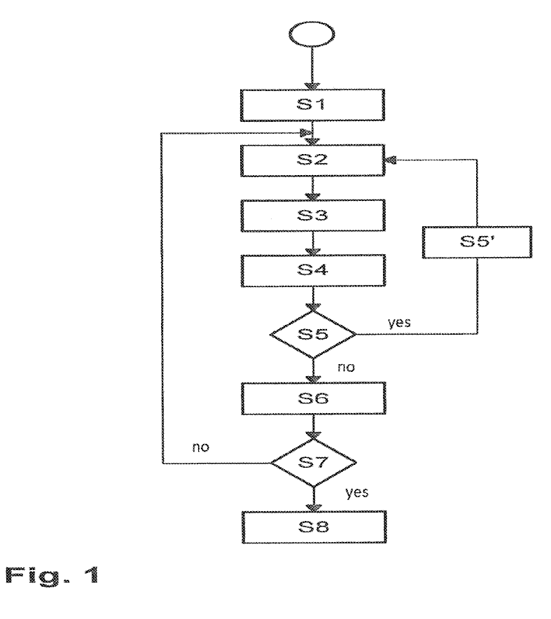

FIG. 1 shows a flow chart of the method in a first embodiment;

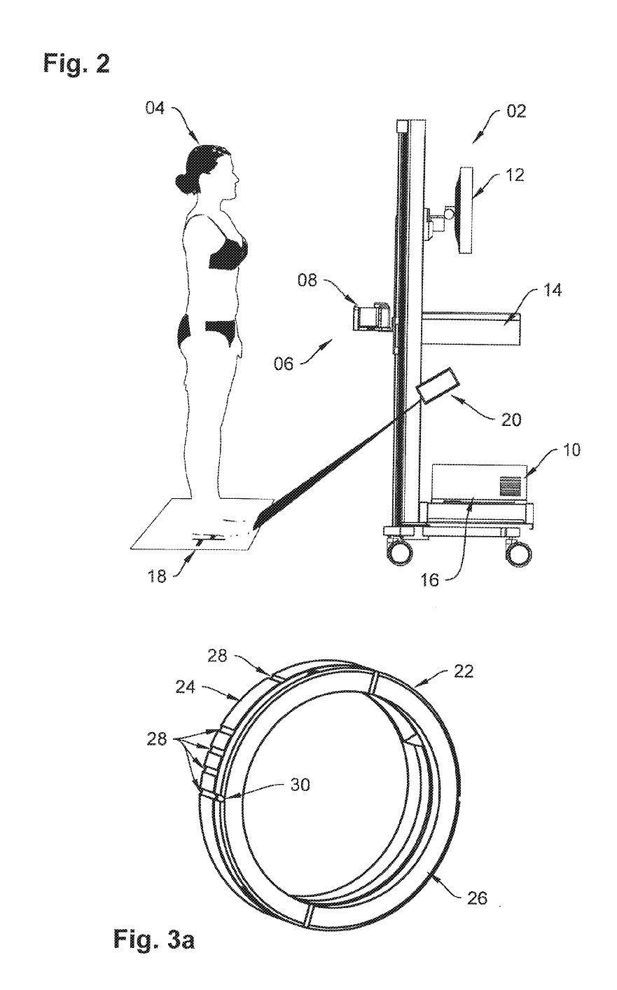

FIG. 2 shows an imaging system according to a first embodiment;

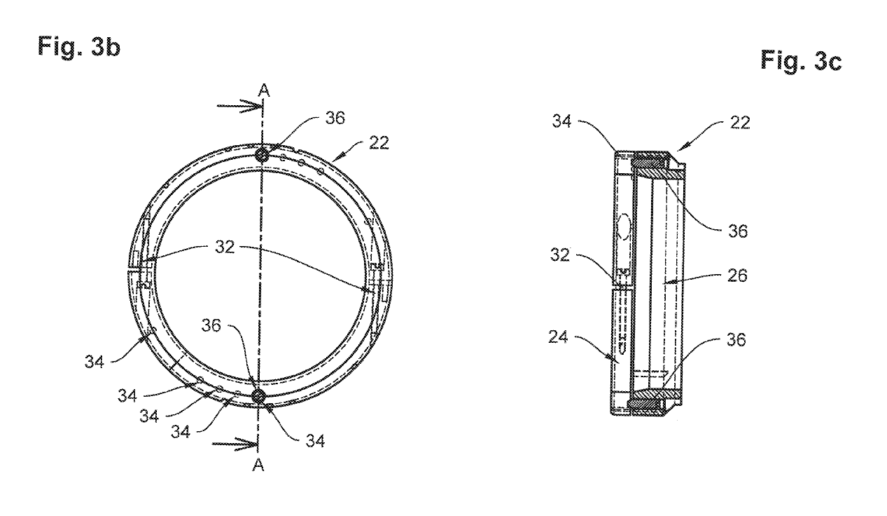

FIG. 3a shows a perspective view of a setting device;

FIG. 3b shows a front view of a setting device according to FIG. 3a;

FIG. 3c shows a cut through the setting device from FIG. 3b along the plane A-A;

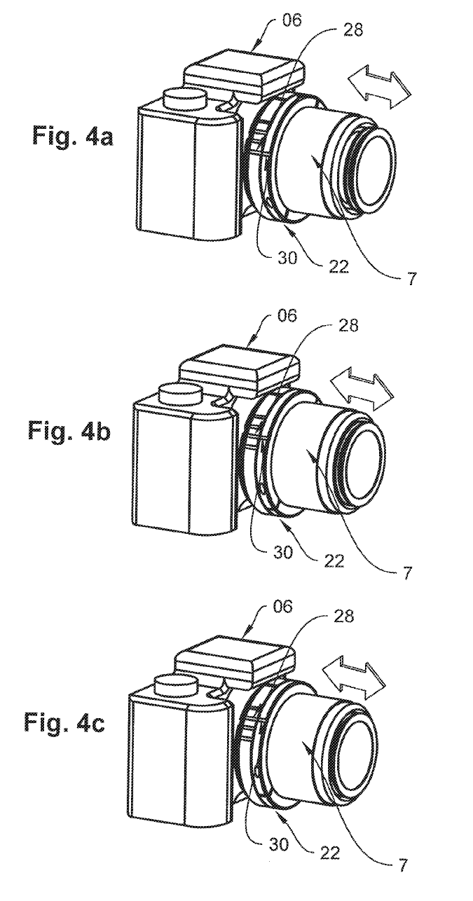

FIG. 4a shows a perspective view of an imaging unit having a setting device in a first setting;

FIG. 4b shows a perspective view of an imaging unit having a setting device in a second setting;

FIG. 4c shows a perspective view of an imaging unit having a setting device in a third setting;

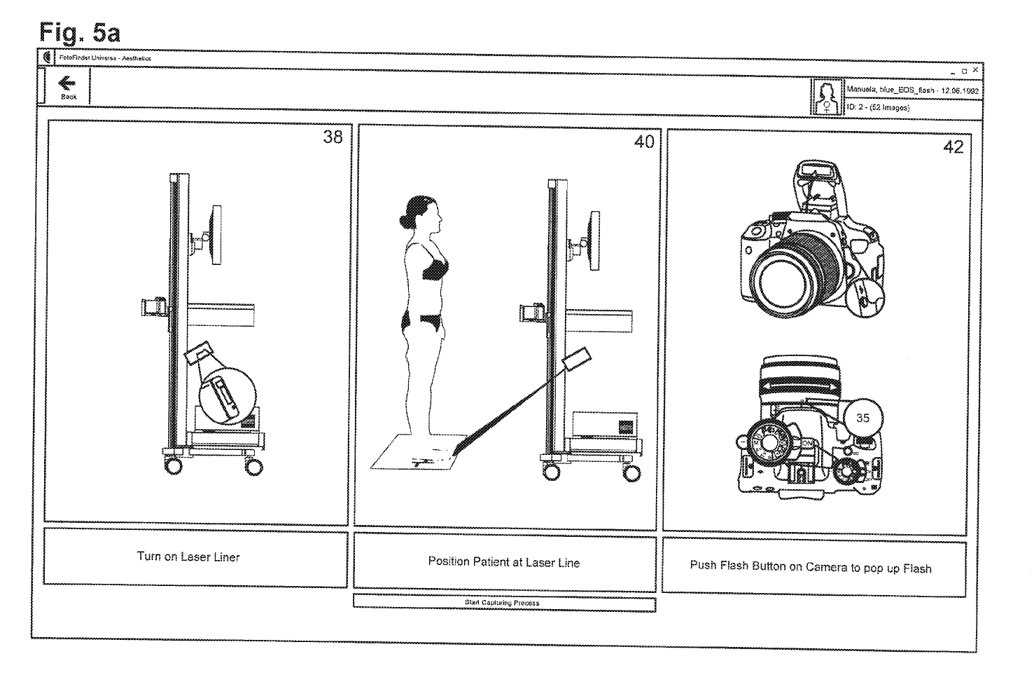

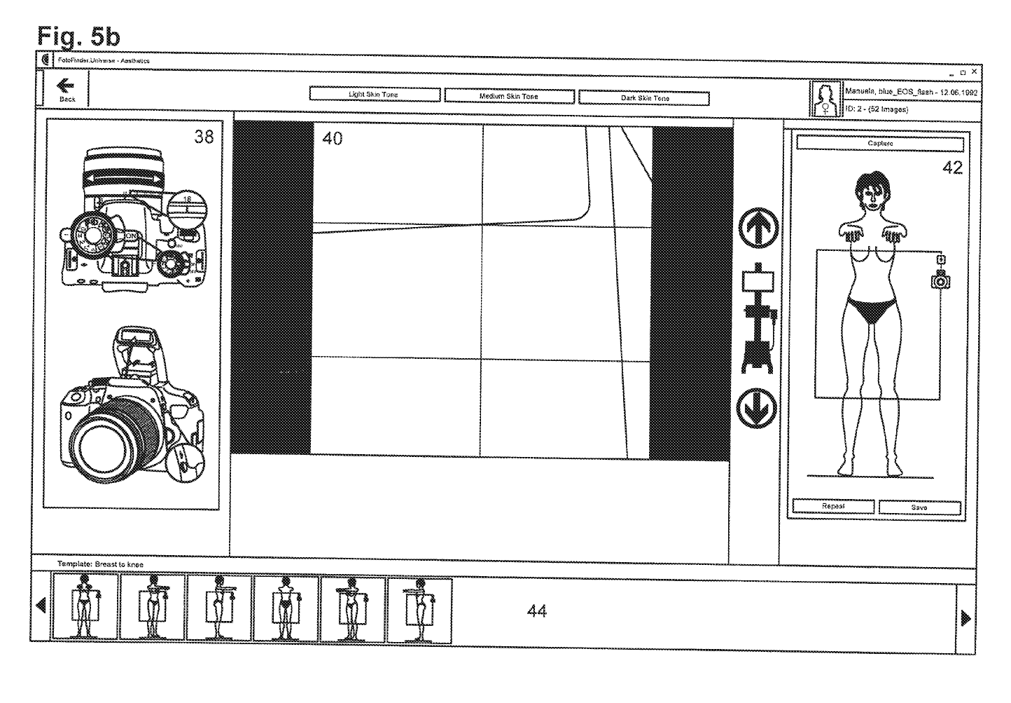

FIG. 5a shows a first exemplary output of an output unit comprising an output of image parameters to be set manually;

FIG. 5b shows a second exemplary output of an output unit comprising an output of image parameters to be set manually; and

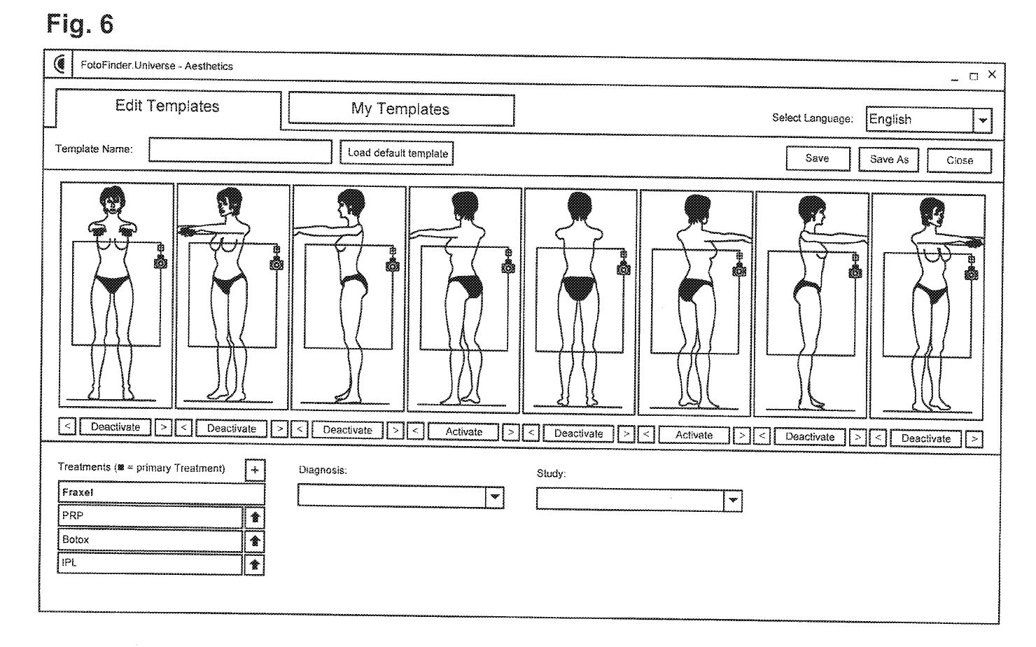

FIG. 6 shows an exemplary output of an output unit for altering a picture series presetting stored in a databank.

FIG. 1 shows a sketched method flow of a specific embodiment of the method according to the invention. After launch, a first method step S1 is executed in which a corresponding picture series presetting is at least selected from a databank. Moreover, depending on whether a basic picture series or a follow-up picture series is generated using the method, object data can be directly or indirectly requested from another databank or be directly input via a setting device. In this instance, moreover, the predetermined image parameters of the picture series presetting can be adjusted by means of the object data within the scope of step S1. The image parameters are adjusted via the object data either while executing the method based on identical object data or only once when executing the method for the first time, the adjusted image parameters then being able to be stored for further runs of the method. Through this, it is ensured that when the object data is input into an input unit, the comparability of the picture series is not prevented by an erroneous input for one or more generated picture series, for example.

In the subsequent method step S2, at least one image parameter to be set manually of the corresponding group of the picture series presetting is output on an output unit, as illustrated in an exemplary manner in FIGS. 5a and 5b, which will be described in further detail below. Moreover, it can also be intended within the scope of method step S2 that at least one remotely settable image parameter is transmitted from the databank and/or the computing unit to a different unit of the imaging system, in particular to the imaging unit and the corresponding setting of the image parameter at the corresponding unit of the imaging system.

Moreover, within the scope of method step S2, a setting device can be used which serves to manually set in a stepwise manner the infinitely manually settable image parameter of the imaging unit. A more detailed description of the setting device as well as its use can be found in the following description of FIGS. 3, 4 and 5.

In the following method step S3, an image is generated for the corresponding set of the picture series presetting by means of the imaging unit. Subsequently thereto, the generated image is transmitted to the computing unit in method step S4.

Subsequently, the image is evaluated by means of the computing unit in method step S5 with respect to at least one manually set image parameter.

In the embodiment of the method illustrated in FIG. 1, it is intended that in the event that the evaluation should yield that the at least one manually set image parameter does not correlate to the output of the image parameter to be set in method step S2, a corresponding output is carried out in method step S5 by means of the output unit in method step S5′ on the one hand and the method additionally jumps back to method step S2. Accordingly, a loop between method steps S2 and S5 will run for a group of a picture series presetting until the evaluation in method step S5 no longer yields a deviation between the at least one output image parameter to be set manually and the actual manually set image parameter.

As soon as no such deviations are yielded as a result of the evaluation, the method is continued with method step S6 by saving the generated images and by a corresponding automatic naming process taking place based on the available data.

In a subsequent method step S7, it is checked by means of the computing unit whether the last generated and saved image correlates to a last group of the picture series presetting. Is this the case, then the method is terminated with method step S8. However, should it be determined that the last generated and saved image is not the last group of the present picture series presetting, then the method is continued with method step S2, the processes described above from method step S2 itself are executed, where necessary, for the next or the following group of the picture series presetting. In other words, this means that each new image parameter to be manually set can be output according to the new group of the picture series presetting and a corresponding automatic setting can be executed if necessary.

FIG. 2 illustrates an imaging system 02 according to the invention as well as an object 04 to be imaged. The imaging system 02 serves for generating several picture series or rather series of images of objects or a part of the object 04 with a temporal delay. The system 02 comprises an imaging unit 06 which is realized as a commercially produced digital camera for example. The imaging unit 06 is arranged at a displacement unit 08 by means of which the imaging unit 06 can be displaced in height. It should be noted, as an example, that the height of the imaging unit is also an image parameter which is set manually or remotely within the scope of the method according to the invention.

The system further comprises a computing unit 10, an output unit 12 and an input unit 14. Moreover, a storage unit 16 is integrated into the computing unit 10, said storage unit 16 being configured for storing at least one databank. For this purpose, it is intended that at least one databank is available in the storage unit 16, said databank comprising a plurality of picture series presettings having, in turn, a plurality of predefined groups of image parameters for an object. Furthermore, the storage unit 16 is configured for storing generated images. The input unit is configured for selecting a picture series presetting. The imaging unit 06 is configured for generating images of the object 04 and for transmitting to the computing unit 10 and/or the storage unit 16.

The output unit 12 is configured for outputting at least one image parameter to be set manually of the corresponding group, and the computing unit 10, moreover, is configured for evaluating an image with respect to at least one image parameter to be set manually of the group of the picture series presetting. Furthermore, the output unit 12 is configured for outputting a notification should the evaluation by means of the computing unit 10 yield that the at least one image parameter to be set manually does not correlate to the output of the corresponding image parameter on the output unit 12.

Furthermore, the system comprises a foot mat 18 on which the object 04 is brought into position, said foot mat 18 or its positioning with respect to the imaging system 02 being able to be detected by means of a measuring device 20. The measuring device 20 can be realized as an optical device, in particular as a laser measuring device. It can be intended that every time the method according to the invention is executed in an initial method step, the arrangement of the foot mat 18 determined by the measuring unit 20 with respect to the imaging system is used for adjusting the corresponding image parameters of all groups of picture series presettings until a renewed altered orientation between the measuring device 20 and the foot mat 18 is detected.

FIG. 3a illustrates a perspective view of a setting device 22. The setting device 22 comprises a first ring element 24 as well as a second ring element 26, both ring elements 24 and 26 being connected so as to be arranged coaxial and rotatable to each other. Moreover, the first ring element 24 comprises a plurality of selection markings 28. A setting marking 30 is moreover arranged on the second ring element 26. The setting device 22 is configured such that it can be arranged at an imaging unit 06 or rather a focusing optics of an imaging unit 06 in such a manner that a stepwise setting of the focal length is enabled at an imaging unit 06 with an infinitely settable focal length.

This functionality of the setting device 22 can be seen in FIG. 3b. This figure shows that the setting device 22 comprises fixing means 32 in the first and/or second ring element 24 or 26, respectively. The fixing means 32 according to FIG. 3b are realized as screws and corresponding thread holes, which enable influencing the inner diameter of the ring elements and a force fit resulting therefrom on parts of the imaging unit or its focusing optics. Alternatively to the fixing means 32, however, the setting device 22 can also be realized such that the setting device 22 or rather the two ring elements 24 and 26 can self-lock on the imaging unit. Furthermore, it is particularly preferred if the setting device comprises calibrating means which enable that the relative arrangement of the ring elements, in which the setting markings 30 are each arranged in extension to a selection marking 28, correlates to determined value of the focal length of the imaging unit. Thus, it is ensured that a relative movement of the ring elements 24 and 26 leads to a determined focal length of the imaging unit, said setting marking 30 and a selection marking 28 being aligned flush to each other.

In order to further enable that the image parameter to be set manually of the focal length of the imaging unit 06 can be set stepwise by means of the setting device 22 even though the imaging unit 06 is provided with an infinitely settable focusing optics 07, the ring elements 24 and 26 can be provided with interacting locking elements 34 and 36. FIG. 3b illustrates, for example, that one of the ring elements 24 or 26 comprises a plurality of indentations 34 and the corresponding other ring element 24 or 26 comprises corresponding engaging elements 36 which are arranged such that they can detachably engage into the indentations 34 when the ring elements 24 and 26 move relative to each other.

The engaging elements 36 can, for example, be realized as spring-loaded bolts which can be pressed against the ring element 24 or 26 comprising the indentations 34 by means of the spring load or a comparable force effect via an elastic element so that the ring elements 24, 26 engage or rather lock when the engaging elements or rather the bolts 36 and the indentations 34 overlap as a result of a relative movement of the ring elements 24 and 26 to each other. For this purpose, it can be intended that the engaging elements 36 and the indentations 34 are arranged to each other in such a manner that the ring elements 24 and 26 lock or engage in the relative positions in which a flush arrangement of the selection markings 28 to the setting marking 30 is available.

In FIG. 3c, the cut through the plane A-A of the setting device 22 from FIG. 3b is illustrated. In FIG. 3b, the fixing element 32 is illustrated again, said fixing element 32 allowing fixing the setting device 22 to the imaging unit 06. Moreover, diametrically opposed indentations 34 in the first ring element 24 and engaging elements 36 in the second ring element 26 interacting therewith are illustrated.

FIGS. 4a to 4c illustrate an imaging unit as can come to use in the method according to the invention and the imaging system according to the invention. The imaging unit 06 comprises a focusing optics 07. Moreover, the imaging unit 06 comprises a setting unit 22 which enables executing a stepwise setting of the focal length as an image parameter, even though the focusing optics 07 is an optics having an infinitely settable focal length. As is illustrated in FIGS. 4a to 4c, the setting marking 30 is aligned flush with one of the selection markings 28 for setting a determined focal length of the focusing optics 07 of the imaging unit 06, whereby the setting device 22 simultaneously engages or rather locks, as is described in reference to FIG. 3.

For this purpose, it can be intended that the selection markings 28 comprise different embodiments, such as a different design color, in order to allow being able to more easily identify the corresponding correct setting position of the setting device 22. Accordingly, it can be intended in the method according to the invention, for example, that the output of the at least one image parameter to be set manually comprises the colored design of the selection marking 28 so that the user can deduce more easily how the setting marking of the setting device 30 is to be positioned regarding the selection markings 28.

FIG. 5a illustrates an exemplary output by means of the output unit 16, which further describes the output in method step S2 of the method according to the invention. The illustration of FIG. 5a essentially illustrates a left segment 38, a middle segment 40 and a right segment 42. The segments 38 to 42 are overall a part of an output which is intended to facilitate using the imaging system 02 or rather the execution of the method for the user. In the left segment 38 as well in the middle segment 40, pictorial as well as written instructions are displayed for the user in order to initiate executing the method.

Moreover, in the right segment 42, two image parameters to be set manually are output. The first image parameter to be set manually is for activating the flash of the imaging unit. The second image parameter to be set manually is for setting the focal length to 35 mm The first image parameter to be set manually is depicted graphically as well as in written form on the right segment 42. The second image parameter to be set manually is depicted only pictorially on the right segment 42 of FIG. 5a. Through the output, as illustrated in an exemplary manner in FIG. 5a, the correct use of the imaging system is facilitated because it is ensured together with the method according to the invention that comparable or reproducible picture series are generated by means of the imaging system 02.

FIG. 5b shows an alternative output as can also be illustrated during method step S2 on an output unit 16 of the imaging system 02. Beside a left segment 38, a middle segment 40 and a right segment 42, the shown illustration also comprises a lower segment 44. The left segment 38 of FIG. 5b essentially corresponds to the right segment 42 of FIG. 5a and serves for outputting two image parameters to be set manually, namely activating the flash of the imaging unit and setting the focal length of the imaging unit. The right segment 42 comprises another output of an image parameter to be set manually. This is the area to be selected of the object 04 to be imaged.

In the right part of the right segment 42, a pictorial depiction of an exemplary object 04 to be imaged and a corresponding representation of the partial area to be selected are illustrated. Moreover, in the left part of the left segment 42, a schematic illustration of the imaging system 02 is illustrated.

The arrows arranged above and below these schematic illustrations can comprise different functions. On the one hand, they can merely serve as an indication to users that the imaging unit 06 has to be displaced by means of the displacement unit 08 such that the part illustrated in the right part of the right segment 42 of the object to be imaged is captured by the imaging unit 06, which can be verified via the control window in the middle segment 40, for example, in which window the actual imaging area of the imaging unit 06 is illustrated.

Alternatively, it can also be intended that the arrows in the right segment 42 of FIG. 5b are realized as control elements, by means of which the imaging unit 06 of the imaging unit 02 can be displaced via the displacement unit 08 of the imaging unit 02 when a certain user input is given. The reason why this possibility is explicitly mentioned is because it clarifies what is deemed to be an image parameter to be set manually in the scope of the present invention. An image parameter to be set manually can also be, as described in the preceding example of FIG. 5b, set indirectly by a user by using corresponding technical setting means. Indicative for being deemed an image parameter to be set manually is that at least any one of the settings is required to be activated by a user.

The lower segment 44 in the exemplary output of FIG. 5b illustrates a picture series presetting, comprising a plurality of predefined groups of image parameters of the object. The different groups of image parameters are elucidated in the example of the lower segment 44 of FIG. 5b mainly through the image parameters of the different spatial orientations of the object regarding the imaging unit. Furthermore, however, it can also be intended that another plurality of image parameters not arising from the pictograms in the lower segment 44 can be different to each other for the corresponding setting.

FIG. 6 shows an exemplary output of an output unit 16 of the imaging system 02, which describes influencing or altering a picture series presetting. Before executing the method for the first time for a certain object or when executing the method for generating a basic picture series, a corresponding picture series presetting can be adjusted. Individual groups of image parameters can, for example, be activated or deactivated in order to slim down the method, as can be taken from the exemplary illustration of FIG. 6. Accordingly, only images for those groups of image parameters are generated or evaluated during the following execution of the method for generating a follow-up picture series which are activated in the picture series presetting.

The user can generate new picture series presettings on their own by means of a comparable output, as illustrated in FIG. 6, and a corresponding interaction or input dialog and output dialog with the user.

1. A method for generating a series of images of objects, each having predetermined image parameters, by means of a partly automated imaging system (02) comprising a data bank, an imaging unit (06), a computing unit (10), a storage unit (16) and an output unit (12), comprising the following method steps:
a) selecting a picture series presetting comprising a plurality of predefined groups of image parameters of an object from a databank (S1);
b) outputting at least one image parameter to be set manually of the corresponding group on the output unit (12) (S2);
c) generating an image by means of the imaging unit (06) (S3);
d) transferring the image to the computing unit (10) (S4);
e) evaluating the image in regard of at least one manually set image parameter by means of the computing unit (10) (S5);
f) outputting a notification by means of the output unit (12) (S5′) should the evaluation yield that the at least one manually set image parameter does not correlate to the output of the image parameter to be set from method step b); and
g) repeating method steps b) to f) for each group of the picture series presetting.

2. The method according to claim 1,
characterized in that
method step g) also comprises saving the image in the storage unit (16).

3. The method according to claim 1,
characterized by
a method step f1), comprising repeating method steps b) to e) for the corresponding group of the picture series presetting should the evaluation yield that the at least one manually set image parameter does not correlate to the output of the image parameter to be set from the method step b).

4. The method according to claim 1,
characterized in that
saving the image in a storage unit (16) comprises automatically generating a filename and/or metadata for the image.

5. The method according to claim 1,
characterized by
a method step b1), comprising using a setting device (22) for a stepwise manual setting of an infinitely settable image parameter of the imaging unit (06).

6. The method according to claim 1,
characterized in that
method step b) also comprises transferring at least one remotely settable image parameter from the databank to a unit of the imaging system (02), in particular the imaging unit (06), and correspondingly setting the image parameter on the part of the unit, in particular the imaging unit (06).

7. The method according to claim 1,
characterized in that
method step a) comprises selecting object data from another databank and/or inputting object data into an input unit (14) of the imaging system (02), the image parameters of the picture series presetting being adjusted in dependence of the object data.

8. An imaging system (02) for generating a series of images of an object, each having predetermined image parameters, comprising a databank, an imaging unit (06), a computing unit (10), a storage unit (16) and an output unit (12), said databank comprising at least one picture series presetting comprising a plurality of predefined groups of image parameters of an object and the input unit (14) being configured for selecting a picture series presetting, said imaging unit (06) being configured for generating images of the object and for transferring them to the computing unit (10) and/or the storage unit (16), and said storage unit (16) being configured for storing generated images,
characterized in that
the output unit (12) is configured for outputting at least one image parameter to be set manually of the corresponding group, the computing unit (10) being configured for evaluating an image in regard of at least one image parameter to be set manually of the group and
the computing unit (12) is configured for outputting a notification should the evaluation by means of the computing unit (10) yield that the at least one manually set image parameter does not correlate to the image parameter to be manually set on the output unit (12).

9. The imaging system (02) according to claim 8,
characterized in that
the computing unit (10) is configured for generating and for saving filenames and/or metadata in dependence of the picture series and/or the group of the picture series and/or object data and/or information input into an input unit (14) in the storage unit (16) with the images.

10. The imaging system (02) according to claim 8,
characterized in that
the imaging system (02) comprises a setting device (22) which is arranged on the imaging unit (06) and enables a stepwise setting of an infinitely settable image parameter.

11. The imaging system (02) according to claim 10,
characterized in that
the setting device (22) is arranged such on the imaging unit (06) that setting the focal length on a focusing optics of the imaging unit (06) in a stepwise manner is enabled using an infinitely settable focusing length.

12. The imaging system (02) according to claim 8,
characterized in that
the imaging system (02) comprises at least one transmitting means with which at least one image parameter, which can be set remotely, is transmitted to a unit of the imaging system (02).

13. The imaging system (02) according to claim 12,
characterized in that
the imaging system (02) comprises at least one setting means for remotely setting an image parameter, said setting means being connected to a transmitting means so as to be able to receive the corresponding remotely set image parameter.

14. The imaging system (02) according to claim 8,
characterized in that
the computing unit (10) is configured for adjusting the image parameter to a picture series presetting using object data.
